(19) United States
(12) Patent Application Publication
(10) Pub. No.: US 2004/0073276 A1
(43) Pub. Date: Apr. 15, 2004

(54) Programming system for medical devices, a server for such a system and a method for managing the system

(76) Inventor: Eric Samuelsson, Jarfalla (SE)

Correspondence Address: SCHIFF HARDIN, LLP, PATENT DEPARTMENT, 6600 SEARS TOWER, CHICAGO, IL, 60606-6473, US

(21) Appl. No.: 10/451,044

(22) PCT Filed: Dec. 18, 2001
(86) PCT No.: PCT/SE01/02822

(30) Foreign Application Priority Data
Dec. 22, 2000 (SE) 0004843-9

(51) Int. Cl. A61N 1/08

(52) U.S. Cl. 607/60

(57) ABSTRACT

In a system for managing the programming of an implantable medical device, a programmer communicates with the implantable medical device via a transceiver, and also communicates with a remote server via a network. The programmer includes a machine-readable code module that interrogates the implanted device to obtain identifying information associated with the implanted device, and the programmer transmits this identifying information, as well as programmer identifying information, to the remote server via the network. Based on this identifying information, the remote server selects an operating program for the programmer and/or the implanted device, and transmits the operating program to the programmer for appropriate installation.

### BACKGROUND OF THE INVENTION

1. Field of the Invention

The invention relates to implantable medical devices and specifically to programming systems for implantable medical devices.

2. Description of the Prior Art

Implantable medical devices, such as cardiac stimulators, drug pumps, neurostimulators and the like operate autonomously to perform a variety of medical functions. They typically comprise a software controlled processor, which commands the various sensing and stimulating functions. The device, or rather its software, should be configured to operate in accordance with a patient's diagnosis and also with the needs of medical practitioners who oversee the patient. In some cases, the implanted device may be able to store information concerning the device operation or the patient's response that is useful for assessing ongoing treatment. The configuration and reconfiguration of an implanted device as well as the accessing of any stored data are achieved using a programmer, which generally communicates with the implanted device through a telemetry link. The programmer is also a software controlled machine.

For a given hardware platform, the operating possibilities of an implantable device are limited to the controlling software of the device itself and of the programmer. An implantable device generally leaves the factory with a current software version installed. A programmer will be used to program several different devices and may be utilized over a number of years. It is thus likely that devices that are programmed by a single programmer will run different versions of operating software. If a programmer is to be able to exploit specific features of a device software, it must run compatible programmer software. Accordingly, each time a new device software appears, a compatible programmer software version must be installed in the programmer by a field technician. The operator is thus obliged to maintain different versions of programmer software and also be aware of which software is required for programming which implanted device. This is a cumbersome and costly process both for the vendor and the operator.

There is thus a need for simplifying the programming of implantable medical devices.

### SUMMARY OF THE INVENTION

The above need is met in a programming system for an implantable medical device that includes a programmer with a transceiver for communicating with implanted medical devices and a memory. The programmer further has a network connection for establishing communication with a remote server through a network and a first machine readable code module arranged to interrogate an implanted device via the transceiver to obtain identifying information associated with the implanted device, transmit the device identifying information and programmer identifying information to the remote server, receive operating software for programming the implanted medical device from the server and install the in the programmer.

By means of this arrangement, the need for the operator to recognize and utilize the required or most recent software for operating the programmer is obviated. The server takes over the management of the operating software modules and by using the identifying information is simultaneously able to track which programmers and medical devices are running which operating software modules.

The functions of the medical devices and programmers thus can be changed and updated easily without the intervention of a field engineer, moreover the operator will always be certain that the correct programming software is being used for each implanted device.

Preferably the first machine readable code module is further arranged to receive machine readable code for operating the device and transmitting the machine readable code for operating the device to the device for installation.

With this arrangement, the programmer machine readable code module does not need to be full fledged operating machine readable code. Providing it can enable communication with the device and server and also read and transmit the identifying information, the required full programming software can be identified and downloaded from the server.

In a preferred embodiment, the implantable medical device includes a hardware platform and an installed operating software module, wherein the first machine readable code module is arranged to read and transmit to the server information identifying said medical device, said installed software and possibly said hardware platform. By separately identifying the device and the device operating software, the server can determine which, if any, additional software is required by the device.

The same information preferably is also provided for the programmer, such that the programmer includes a hardware platform and an installed programming software module, wherein said first machine readable code module is arranged to read and transmit to the server information identifying the programmer, the installed software and possibly the hardware platform.

The invention also is directed to an implantable medical device that includes a memory for storing machine readable code, a transceiver for communicating with a programmer and further a first machine readable code module arranged to communicate through the transceiver identification information associated with the device to a programmer. The first machine readable code module is further arranged to enable the device to receive operating machine-readable code from the programmer and to install the operating code to enable operation of the code to control the device. The ability of the machine readable code running on the device to install new or additional operating software greatly improves the operating flexibility of the device.

In accordance with a further aspect of the invention, the above need is met by a server for communicating with programmers of implantable medical devices through a network. The server includes storage means for storing identifying information associated with implantable medical devices, programmers and software modules for the operation of the medical devices and programmers. Furthermore, the server is configured to process software requests from a programmer, and download to the programmer software modules for installation on the programmer and/or a medical device.

The invention also concerns a method for managing a system for programming an implantable medical device, wherein the system includes an implantable medical device, a programmer adapted to communicate with the implantable medical device and a remote server, wherein the programmer is adapted to communicate with the remote server though a network. The method includes the steps of: interrogating the medical device using the programmer to obtain device identifying information, transmitting the device identifying information and information identifying the programmer to the server, downloading operating software for the programmer and/or the medical device to the programmer for installation on the programmer and/or the device on the basis of the identifying information.

Preferably, the interrogation step includes interrogating the medical device to obtain information identifying the device and information identifying an operating software module installed on the device, while the transmitting step preferably includes transmitting information identifying the programmer and information identifying an operating software module installed on the programmer.

In a preferred embodiment of the invention, the method includes the server performing the steps of: determining a required operating software module for the programmer and/or the implanted medical device, comparing identifying information of the required software module with the transmitted software identifying information and downloading the required software if the transmitted software identifying information is not the same as the required software identifying information. In this way, the system can ensure simply and rapidly that both a programmer and the medical devices monitored using the programmer are utilizing the most recent and compatible software.

### BRIEF DESCRIPTION OF THE DRAWINGS

FIG. 1 is a schematic illustration of a system for programming an implantable medical device constructed and operating in accordance with the principles of the present invention.

FIG. 2 is a flowchart for a method for programming a programmer and/or an implantable medical device in accordance with the present invention.

### DESCRIPTION OF THE PREFERRED EMBODIMENTS

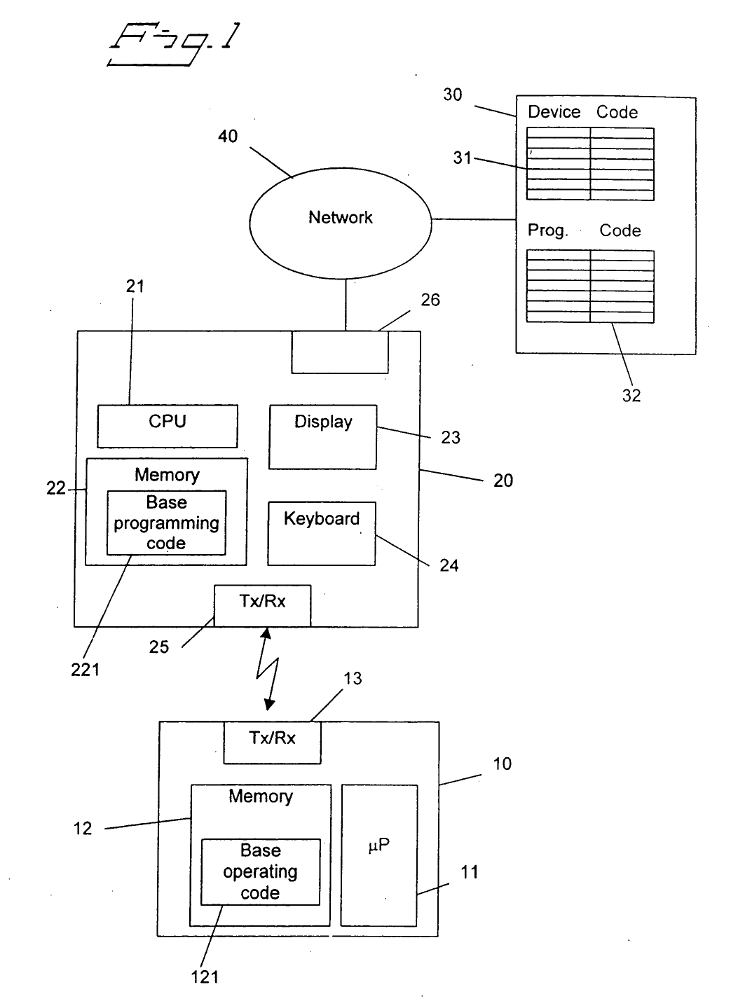

In general, when a pacemaker or other implantable medical device is purchased, it contains the most recent version of operating software. After being implanted in a patient, the device must be configured to suit the patient's needs. This is accomplished using a programmer. A single programmer will typically configure and monitor various different devices having different configurations. For example, the programmer may be used to configure and monitor devices, which have an identical hardware platform to the newly implanted device but which run a different version of the operating software. If a programmer is to configure and monitor an implanted medical device correctly, it is necessary for the programmer software to be compatible with the functions of the device operating software. Currently, whenever a new version of device operating software is produced and installed in new devices 10, the programmers likely to program any new devices must be updated with a compatible programmer software by a field engineer. Moreover, the programmer must hold programming software versions compatible with the operating software installed in each device served by the programmer. The operator must also be aware of which programmer software must be used with which device and ensure that the correct programmer software is used in each case. This situation is simplified in accordance with the invention by using the arrangement depicted in FIG. 1.

FIG. 1 shows a system for programming implantable medical devices. In the illustrative embodiment, an implantable medical device 10 is a cardiac stimulator or pacemaker. This device is implanted in the conventional manner beneath the skin in the chest of a patient and is connected to one or more stimulating and/or sensing electrodes (not shown) that are embedded in the heart tissue. As illustrated in FIG. 1, the device 10 includes at least the following elements: a central processor 11, such as a microprocessor (μP) minicomputer or the like, for controlling the operation of the device, a memory 12 and a transmitter/receiver module 13 for transmitting and receiving data to and from a programmer 20 via a telemetry link in the conventional manner.

Turning now to the programmer 20, it is apparent from FIG. 1 that the programmer 20 also includes some form of processing means for controlling its operation in the form of a central processing unit CPU 21. The programmer 20 also includes a memory 22, some form of display for displaying information about the implanted device 10 or programmer to the operator, a keyboard or other input device, such as a touch-sensitive screen or cursor moving device 24, a transmitter/receiver module 26 for transmitting and receiving information to and from the implanted device 10 through the telemetry link and a network interface module 26 for permitting access to a network 40, and through the network 40 to a server 30.

The network 40 may be any public or private telecommunications network enabling communication and the exchange of data between one or more programmers 20 and a server 30. For example, the network may be a private computer network, the PSTN accessed via a dial-up connection using a modem, or the Internet. It will be appreciated that several programmers 20 will be able to access a single server 30 or group of servers 30 through the network 40.

It will be understood that the illustrated functional and structural organization of the implanted device 10 and programmer 20 is an example only and that modified or additional elements may be present in these elements.

When new device 10 is implanted in a patient, it will not contain a full operating software. Instead the operation is controlled by a base operating machine-readable code module 121 that supports the basic functions of the pacemaker 10 and additionally permits communication with the programmer 20 via the telemetry link. This base software is also able to identify the device

10 to the programmer 20 using the telemetry link. Preferably, each device 10 has a unique identifier that is known to the server 30. The device 10 preferably also will have an identifier for the hardware platform. Finally, any operating software, whether this is the base operating machine-readable code module 121 or full operating software, will also have a unique identifier. These identifiers will also be available for any full operating software versions that may be installed. The base operating code module 121 further enables the device 10 to receive software through the telemetry link and is able to install any new software received this way.

A new programmer 20 will likewise not have a full programming software installed. On delivery it will also run a skeleton software illustrated in FIG. 1 by a base programming machine-readable code module 221 that supports the basic functions of the programmer and in addition allows the programmer 20 to communicate with any implanted device through the telemetry link and to read the device identifiers. In addition, the base programming code module 221 enables communication with the server 30 through the network 40. Specific functions of the base programming code module 221 include the ability to receive and install software received from the server 30 via the network 40 and also transmit or download software to the device 10 for installation. As for the device 10, the programmer 20 has several unique identifiers. These include an identifier for the hardware platform, an identifier for the programming software installed, whether this is the base programming code module 221 or one or more full programming software versions, and finally a unique identifier for the device itself. The base programming code module 221 knows or is able to read these identifiers and transmit these to the server 30.

The server 30 includes a database, represented in FIG. 1 by tables 31, 32, where the unique identifiers relating to the device 10 and programmer 20 are stored, respectively. Moreover, the server 30 identifies for each device 10 and device hardware platform the most recent device operating software, and also identifies the relevant programming software version for the programmer and its hardware platform. Accordingly, when the server 30 receives a request from a programmer 20 identifying a device and a programmer, the server is able to communicate to the programmer the possible device operating software versions and corresponding programmer software versions that are available.

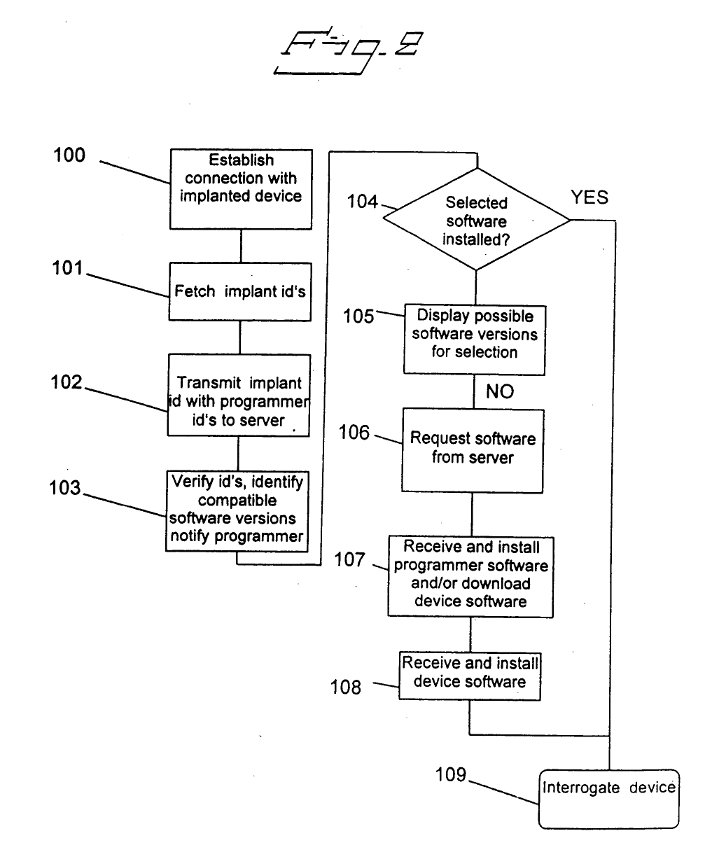

The operation of this system will be described with reference to the flow chart of FIG. 2.

The system operation starts with step 100, in which the programmer 20 establishes a connection with the implanted device 10 through the telemetry link. The base programming code module 221 then fetches the implanted device identifiers from the base operating code module 121 of the device 10 in step 101. As already mentioned these identifiers include the identifier of the current installed operating software, the device identifier and preferably also a hardware platform identifier. If full operating software is not installed in the device, the software identifier will correspond to the base operating code module 121. These identifiers are then transmitted by the programmer 20 to the server 30 susing its base programming code module 221, while in addition the base programming module 221 will also read and transmit the corresponding programming software, programmer and hardware platform identifiers to the server 30 in step 102. In step 103 the server 30 verifies the transmitted identifiers, and if these are correct, identifies the required compatible software modules for operating and programming the implanted device 10. In step 104 the server 30 determines whether the most recent software versions are already installed by comparing the identifiers of possible software modules with the software module identifiers transmitted from the device 10 and programmer 20. If the software is already installed, the server 30 returns as suitable message to the programmer 20, when then commences the standard device programming and monitoring procedure in step 109. If conversely the latest software is not installed in either the programmer 20 or the device 10, the server 30 notifies the programmer 20 of the possible software versions that may be installed, and in step 105 the programmer presents the choice of software modules available to the operator on the display 23 and prompts the operator to make a selection. After the operator has selected the required software modules using the keyboard 24 or other available input device, the programmer 20 sends a request to the server 30 for the selected software modules in step 106. The requested modules are sent by the server 30 through the network 40 and received by the programmer in step 107. The programmer 20 also installs any programming software destined for the programmer on itself and downloads any new operating software to the implanted device 10. In step 108 the implanted device 10 receives and installs the new device software, if any. In step 109, the programmer then commences the conventional programming and monitoring procedure by interrogating the implanted device for the various data.

If both the programmer 20 and implanted device are new with no programming or operating software loaded, respectively, the initial steps 100 to 108 will be performed by the base programming code modules 221 and 121. Otherwise, the currently installed programming or operating software could perform the relevant steps. It will be understood, however, that the base programming and operating code modules 221, 121 may continue to perform certain functions, such as fetching the implanted device identifiers, communicating with the server and installing the downloaded software, while any additional functions required for the full operation of the implanted device or programmer are provided by the additional software obtained through the server.

This arrangement allows more flexibility for the vendor and the operator of the programmer. There is no need to deliver products with the entire software already installed, since the server can identify at any time which software is required for the interaction between the device 10 and programmer 20 and also make this software available. Since all decisions on what software to download are based on the identifiers of the programmer and implanted device, the server may also keep track of the services provided to the different customers. This means that the server may not simply provide the latest compatible software revisions to the programmer and or implanted device. Instead, other information may influence the services provided. For example, the server may permit only prepaid software versions to be downloaded to a particular device identifier. In an alternative operation, the customer, whether the programmer operator or implanted device holder, could be billed following a software update through the server.

If a recall of a particular software revision is required, the server need simply be configured not to issue that software. Furthermore, the server will hold a record of the various devices and/or programmers that have received the software in question, so these customers could be notified, either by a message from the server next time they login or through other means.

Although modifications and changes may be suggested by those skilled in the art, it is the invention of the inventors to embody within the patent warranted heron all changes and modifications as reasonably and properly come within the scope of their contribution to the art.

1. A programming system for an implantable medical device, said system including a programmer (20) with a transceiver (25) for communicating with implanted medical devices (10) and a memory (22), characterised in that said programmer further comprises a network connection (26) for establishing communication with a remote server (30) through a network (40) and
a first machine readable code module (221) arranged to interrogate an implanted device (10) via said transceiver to obtain identifying information associated with said implanted device, transmit said device identifying information and programmer identifying information to said remote server,
receive from said server operating software for execution by said programmer for programming said implanted medical device and install said operating software in said programmer.

2. A programming system as claimed in claim 1, characterised in that said first machine readable code module (221) is further arranged to enable receipt of machine readable code for operating said device by said programmer (20) and
transmission of said machine readable code for operating said device to said device for installation.

3. A system as claimed in claim 1 or 2, characterised in that said implantable medical device includes a hardware platform and an installed operating software module, wherein said first machine readable code module is arranged to read and transmit to said server information identifying said medical device, said installed software and possibly said hardware platform.

4. A system as claimed in any previous claim, characterised in that said programmer includes a hardware platform and an installed programming software module, wherein said first machine readable code module (221) is arranged to read and transmit to said server information identifying said programmer, said installed software and possibly said hardware platform.

5. A system as claimed in any previous claim, characterised in that said implantable medical device is a cardiac stimulating device.

6. A server for communicating with programmers (20) of implantable medical devices (10) through a network (40), characterised in that said server (30) includes storage means (31, 32) for storing identifying information associated with implantable medical devices, programmers and software modules for operation of said medical devices and programmers, said server further being arranged to process software requests from a programmer (20), and download to said programmer software modules for installation on said programmer (20)

7. A server as claimed in claim 6, further arranged to download to said programmer software modules for installation on said medical device (10).

8. A method of managing a system for programming an implantable medical device including an implantable medical device (10), a programmer (20) adapted to communicate with said implantable medical device and a remote server (30), wherein said programmer is adapted to communicate with said remote server through a network (40) said method including the steps of:
interrogating said medical device using said programmer to obtain device identifying information,
transmitting said device identifying information and information identifying said programmer to said server,
downloading operating software to said programmer for installation on said programmer on the basis of said identifying information.

9. A method as claimed in claim 8, further including the step of:
downloading operating software for said medical device to said programmer for installation on said device on the basis of said identifying information.

10. A method as claimed in. Claim 8 or 9, characterised in that the interrogation step includes
interrogating said medical device to obtain information identifying said device and information identifying an operating software module installed on said device.

11. A method as claimed in any one of claims 8 to 10, characterised in that said transmitting step includes transmitting information identifying said programmer and information identifying an operating software module installed on said programmer.

12. A method as claimed in claim any one of claims 8 to 11, characterised by the steps of
in said server, determining a required operating software module for said programmer (20) and/or said implanted medical device (10), comparing identifying information of said required software module with said transmitted software identifying information and downloading said required software if the transmitted software identifying information is not the same as said required software identifying information.
